(19) United States
(12) Patent Application Publication
(10) Pub. No.: US 2005/0003376 A1
(43) Pub. Date: Jan. 6, 2005

(54) Single molecule detection with surface-enhanced raman scattering and applications in DNA or RNA sequencing

(75) Inventors: Katrin Kneipp, Berlin (DE); Harald Kneipp, Berlin (DE); Irving Itzkan, Boston, MA (US); Ramachandra R. Dasari, Lexington, MA (US); Michael S. Feld, Waban, MA (US)

Correspondence Address: Timothy J. Oyer, Ph.D.;Wolf, Greenfield & Sacks, P.C., 600 Atlantic Avenue, Boston, MA, 02210, US

(73) Assignee: Massachusetts Institute of Technology, Cambridge, MA (US)

(21) Appl. No.: 10/723,174

(22) Filed: Nov. 26, 2003

Related U.S. Application Data
(63) Continuation of application No. 10/054,729, filed on Jan. 22, 2002, now abandoned.
Application No. 10/054,729 is a continuation of application No. 09/063,741, filed on Apr. 21, 1998, now abandoned.
(60) Provisional application No. 60/076,310, filed on Feb. 27, 1998.

(51) Int. Cl. C12Q 1/68; G01N 33/543

(52) U.S. Cl. 435/6; 436/518

(57) ABSTRACT

Surface-enhanced spectroscopy, such as surface-enhanced Raman spectroscopy employs aggregates that are of a size that allows easy handling. The aggregates are generally at least about 500 nm in dimension. The aggregates can be made of metal particles of size less than 100 nm, allowing enhanced spectroscopic techniques that operate at high sensitivity. This allows the use of larger, easily-handleable aggregates. Signals are determined that are caused by single analytes adsorbed to single aggregates, or single analytes adsorbed on a surface. The single analytes can be DNA or RNA fragments comprising at least one base.

### RELATED APPLICATIONS

This application is a continuation of U.S. patent application Ser. No. 10/054,729, filed Jan. 22, 2002 (pending), which is a continuation of U.S. patent application Ser. No. 09/063,741, filed Apr. 21, 1998 (abandoned), which claims priority to U.S. provisional application Ser. No. 60/076,310, filed Feb. 27, 1998, all of which are incorporated herein by reference.

### GOVERNMENT FUNDING

This invention was made with government support under Grant Number NIH-5P41-RR02594, awarded by NIH. The government has certain rights in the invention.

### FIELD OF THE INVENTION

The present invention relates to methods for detection of analytes, and more specifically to techniques for the detection of a single analyte by surface-enhanced Raman scattering (SERS) and for sequencing DNA or RNA by using the SERS technique.

### BACKGROUND OF THE INVENTION

The presence of molecules and their ground state electronic, vibrational and corresponding excited state structures can be detected by a variety of spectroscopic techniques. Typically, the molecules are dispersed in a medium such that solvent or other intermolecular actions may affect the measured spectroscopic values. There are several applications, however, that require the detection of a single molecule and the determination of its electronic or vibrational structure. Several difficulties lie in meeting the challenge of single molecule detection, namely (1) finding a way to isolate a single molecule and (2) finding a spectroscopic technique to detect the single molecule and output a signal of sufficient intensity.

A molecule may absorb or emit electromagnetic radiation. Spectroscopy is a technique to monitor this absorbance or emission, and furthermore, the energy of the electromagnetic radiation can provide information on an electronic, vibrational or rotational structure of the molecule. For example, visible radiation can excite an electronic transition, causing the molecule to be promoted to an excited electronic state. Fluorescence occurs when a molecule emits electromagnetic radiation. When a molecule absorbs infrared radiation, a vibrational transition can occur to cause the molecule to be promoted to an excited vibrational state.

A molecule can also scatter radiation. Rayleigh scattering is an elastic collision between a molecule and an incident photon of an energy, hv^{o}, such that the photon is scattered with unchanged energy, hv^{o}. Raman spectroscopy involves an inelastic scattering process in which a molecule having an energy, hv_{υ}, collides with an incident photon energy, hv^{o}, causing the molecule to be promoted to an excited vibrational state and leaving the photon with an energy h(v^{o}−v_{υ}). An incident photon, hv^{o} can also collide with a molecule that already exists in an excited vibrational state of energy, h(v+v_{υ}). The photon obtains a new energy, h(v^{o}+v_{υ}), whereas the molecule is demoted to a ground vibrational state hv. From the Boltzmann distribution, fewer molecules are found in an excited vibrational state and thus the latter scattering event occurs much less rarely. A Raman spectrum consists of two sets of Raman signals termed “Stokes” lines and “anti-Stokes” lines. In the Raman spectrum, Stokes lines are attributed to photons having frequency values of v^{o}−v_{υ}, and anti-Stokes lines result from photons having frequency values, v^{o}+v_{υ}. Because fewer molecules exist in an excited vibrational state, the intensity of anti-Stokes lines is much less than the intensity of Stokes lines. In general, Raman scattering is an inefficient process; only 10⁻⁸ to 10⁻¹⁰ of the intensity of the incident frequency produces Raman scattering.

The intensities of Raman signals are enhanced considerably when the molecules are attached to surfaces of metallic structures having nanometer dimensions. This enhancement is termed “surface-enhanced Raman scattering” (SERS). The surface enhancement involves, in part, electromagnetic radiation inducing an electromagnetic resonance which is confined to the surface, the electromagnetic resonance which in turn enhances a surrounding optical field. For example, when the surface comprises a plurality of spatially isolated particles having a dimension smaller than the wavelength of the applied electromagnetic radiation the resulting electromagnetic resonances are confined to localized areas and are termed surface “plasmons.” The electromagnetic enhancement is particularly effective for colloidal particles.

### DISCUSSION OF THE RELATED ART

Nie et al. report the probing of single molecules adsorbed on nanoparticles by surface-enhanced Raman scattering. Science 1997, 275, 1102-1106. The high enhancement efficiencies were attributed to “hot particles” which are single particles having a dimension of 100 nm to 120 nm. The analytes are subjected to visible resonant radiation, which results in the disappearance or change of the Raman signals after a few minutes of continuous illumination.

U.S. Pat. No. 4,962,037 discloses a method for DNA or RNA base sequencing. Each base within a single fragment of DNA or RNA is tagged with a fluorescent dye having an identifiable characteristic for the base. The bases are then cleaved into a flow stream and identified by laser-induced fluorescence.

U.S. Pat. No. 5,306,403 describes a method and apparatus for analyzing DNA. A SERS label, typically a dye, is attached to DNA fragments and at least one DNA fragment is adsorbed onto a SERS-active media. A SERS spectrum has characteristics which identify the dye label of the DNA fragment.

U.S. Pat. No. 5,674,743 relates to a method and apparatus for automated DNA sequencing. A single nucleotide is incorporated in a fluorescence-enhancing matrix and irradiated to cause fluorescence. The single nucleotide is then identified by its fluorescence.

U.S. Pat. No. 5,351,117 describes a method for identifying a diamond or other specific luminescing minerals. The diamond or mineral is irradiated with a high-frequency modulated radiation. The anti-Stokes radiation emitted from the diamond or mineral is isolated and analyzed.

A powerful application for single molecule detection is found in DNA sequencing. Current methods for DNA sequencing involve the obtaining nucleotides of various sizes, running the fragments through a gel, and analyzing the fragments to observe a pattern of bands from which the sequence can be determined. However, these methods require radioactive labeling or fluorescence tags.

### SUMMARY OF THE INVENTION

The present invention provides systems and techniques for determining the presence of analytes using surface enhanced emission spectroscopy.

In one aspect the invention provides a method for determining the presence of at least one analyte. The method involves providing a sample comprising a plurality of aggregates of size of at least about 500 nm adsorbing a plurality of analytes. The sample is exposed to electromagnetic radiation to cause surface-enhanced emission. Spectral information of the sample is obtained, where at least one spectral line of the information represents a single analyte adsorbed on one of the plurality of aggregates.

Another embodiment involves providing a sample comprising a plurality of aggregates adsorbing a plurality of analytes, where at least one aggregate of the plurality of aggregates comprises a metal cluster of at least seven particles and adsorbs only one analyte. The sample is exposed to electromagnetic radiation to cause surface-enhanced emission, and spectral information is thereby obtained, in which the only one analyte contributes to the spectral information. The spectral information can be a portion of a Raman spectrum, and can be a single line of a Raman spectrum.

In another embodiment a method is provided that involves using a sample comprising a plurality of aggregates adsorbing a plurality of analytes where each aggregate comprises a plurality of metal particles. Each metal particle has a dimension of no more than about 100 nm, and at least one aggregate adsorbs only one analyte. The sample is exposed to electromagnetic radiation to cause surface-enhanced emission, and spectral information is thereby obtained. The only one analyte contributes to this spectral information.

In another embodiment a method is provided in which a sample comprising a plurality of aggregates is exposed to electromagnetic radiation. At least one aggregate adsorbs only one analyte that is free of an emission-enhancing aid. Spectral information is obtained, where the only one analyte contributes to at least one signal of the spectrum.

In another embodiment a method is provided for determining the presence of a single analyte. A sample is provided that comprises a plurality of surfaces, such as surfaces of a plurality of aggregates or multiple surfaces of aggregates immobilized on a substrate. A portion of the plurality of surfaces adsorbs only one analyte. The sample is exposed to electromagnetic radiation to cause it to emit radiation in a manner such that the sample is free of photobleaching.

In another embodiment a method is provided for determining the presence of at least one molecule. At least one molecule is provided and exposed to electromagnetic radiation to cause surface-enhanced Raman scattering. Raman spectral information is obtained and the presence of the at least one molecule is determined from at least one anti-Stokes line.

The invention also provides methods for sequencing at least a portion of DNA or RNA. The method involves cleaving the at least a portion of DNA or RNA into DNA or RNA fragments, wherein each fragment comprises at least one base. Each DNA or RNA fragment is then allowed to become surface-adsorbed. Each fragment is exposed to electromagnetic radiation to cause surface-enhanced emission, and unique surface-enhanced spectroscopic information is obtained which is attributed to each fragment.

In another embodiment a method for general field enhancement is provided. The method involves providing a plurality of aggregates and exposing the aggregates to near infrared radiation. At least one electromagnetic resonance is induced in the plurality of aggregates to cause a surface-enhanced radiation.

In another embodiment a method for selecting a spectral range is provided. A sample is provided and at least one filter is positioned in association with an optical excitation and detection system. The system is free of a spectrograph and the optical excitation system produces electromagnetic radiation in a first range. The sample is exposed to electromagnetic radiation via the system, and a surface-enhanced emission spectrum is obtained that has a second range.

In another embodiment a method for determining the presence of an analyte is provided. A sample is provided comprising a rough metal film including a plurality of protrusions and indentations. A plurality of analytes is adsorbed on a surface of the film. The sample is exposed to electromagnetic radiation to cause surface-enhanced emission, and unique spectral information is obtained attributed to the single analyte.

In another aspect a system is provided. In one embodiment the system includes a sample, a source of electromagnetic radiation positioned to irradiate the sample, and a detector positioned to detect surface-enhanced emission from the sample. The sample includes analytes adsorbs on aggregates where the aggregates have a minimum dimension of about 500 nm. In another embodiment a similar system is provided in which the aggregates need not necessarily have a minimum dimension of 500 nm but are made of particles of no more than about 100 nm.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE DRAWINGS

FIG. 1 illustrates a schematic of a Raman spectrum displaying Stokes and anti-Stokes lines and one line attributed to Rayleigh scattering;

FIG. 2 illustrates a schematic of a prior art surface-enhanced spectral system;

FIG. 3 illustrates a schematic of a system of the invention without a spectrograph;

FIG. 4 shows 100 SERS spectra collected from a 30 pL probed volume containing an average of 0.6 crystal violet molecules displayed in the time sequence of measurement where each spectrum is acquired in 1 s;

FIG. 5 shows peak heights of (a) the 1174 cm⁻¹ line for the 100 SERS spectra shown in FIG. 4 and (b) of the 1174 cm⁻¹ line for 100 spectra from a sample without crystal violet, to establish the background level; (c) of the 1030 cm⁻¹ line for 100 spectra measured from 3 M methanol;

FIG. 6 shows statistical analysis of (a) 100 “normal” Raman measurements at the 1030 cm⁻¹ line for 10¹⁴ methanol molecules; (b) 100 SERS measurements of the 1174 cm⁻¹ line of six crystal violet molecules in the probed volume where the solid lines are Gaussian fits to the data; (c) 100 SERS measurements of the 1174 cm⁻¹ line for an average of 0.6 crystal violet molecules in the probed volume where the peaks reflect the probability to find just 0, 1, 2 or 3 molecules in the probed volume;

FIG. 7 shows (a) an electron micrograph of typical SERS-active colloidal silver clusters; (b) an absorption spectrum of SERS-active silver clusters in aqueous solution; (c) an absorption spectrum of a 10⁻⁶ M solution of pseudoisocyanine in methanol;

FIG. 8 shows 100 SERS and Raman spectra, respectively, collected from 0.9 pseudoisocyanine molecules and 10¹³ methanol molecules in the probed volume, displayed in the time sequence measurement where each spectrum was collected in 1 s;

FIG. 9 shows anti-Stokes SERS spectra, collected from the same sample as FIG. 8 where each spectrum was collected in 1 s;

FIG. 10 shows typical spectra measured from a sample which contains 0.5 pseudoisocyanine molecules and 10¹³ methanol molecules (*) in the probed volume where the spectra represent approximately 0, 1 or 2 pseudoisocyanine molecules in the probed volume;

FIG. 11 shows a statistical analysis of 200 spectra at (a) 1360 cm⁻¹ and (b) 1450 cm⁻¹ where both (a) and (b) are measured from a sample which contains 0.5 pseudoisocyanine molecules and 10¹³ methanol molecules and the data were fit by the sum of three Gaussian curves (solid line) which reflect the Poisson distribution for detecting 0, 1 or 2 pseudoisocyanine molecules in the actual measurement and the methanol Raman signal shows the expected Gaussian statistics;

FIG. 12 shows SERS spectra measured at 407 nm from a crystal violet solution having a concentration of (a) 10⁻⁶ M on isolated spheres and (b) 10⁻⁸ M on small clusters;

FIG. 13 shows Stokes and anti-Stokes SERS spectra and signal ratios (table) measured at 10⁶ W/cm² 830 nm excitation from a 10⁻⁸ M crystal violet solution attached to silver clusters having a dimension of 5 μm and 100-500 nm (100-500 nm particles not seen in FIG. 13) and where the anti-Stokes to Stokes ratio of toluene Raman scattering establishes the Boltzmann population for the estimate of the effective SERS cross section;

FIG. 14 shows near infrared-SERS Stokes and anti-Stokes spectra of the order of hundreds of molecules of (a) adenosine monophosphate and (b) adenine both adsorbed on 100-150 nm sized clusters and (c) of adenine adsorbed on a cluster having a dimension of about 8 μm;

FIG. 15 shows typical SERS Stokes spectra representing approximately “1” (top), “0” (middle), or “2” (bottom) adenine molecules in the probed volume where the collection time is 1 s and at the excitation radiation is 80 mW near infrared radiation; and

FIG. 16 shows a statistical analysis of 100 SERS measurements of (a) an average of 1.8 adenine molecules in the probed volume where the x-axis is divided into bins with widths of 5% of the maximum of the observed signal, the y-axis displays the frequency of the appearance of the appropriate signal levels in the bin, the experimental data were fit by the sum of three Gaussian curves (solid line) whose areas are roughly consistent with a Poisson distribution for an average number of 1.3 molecules and which reflects the possibility to observe 0, 1 or 2 (or 3) adenine molecules in the actual measurement and (b) for 18 adenine molecules in the probed volume performed in analogy to FIG. 16(a) where the solid line represents a Gaussian fit to the data.

### DETAILED DESCRIPTION

In one aspect, the present invention resides primarily in the discovery of certain dimensions of surfaces on which analytes immobilized for spectral determination can reliably produce signals attributable to single analyte molecules. Ranges of aggregate sizes, and sizes of particles that make up aggregates, and ranges of sizes of features (indentations and protrusions defined by aggregates of the invention at surfaces) on surface films, have been identified that facilitate spectral detection of single molecules. It is a feature of the invention that near-infrared (near-IR) electromagnetic radiation can be used in spectroscopy in some embodiments, under certain conditions of aggregate size, particle size, or surface feature size, for detection of a single analyte. Another aspect involves a technique in which at least a portion of DNA or RNA is into DNA or RNA fragments where each fragment is allowed to become surface-absorbed and probed by spectroscopy. In this aspect a unique signal is obtained attributed to a single, isolated DNA or RNA fragment, which comprises at least one base. The fragment can be labeled or unlabeled. It is a feature of the invention that unlabeled fragments can be detected.

It is a feature of the invention that a single analyte can be measured. The DNA fragment, the RNA fragment, and each DNA and RNA base are examples of a single analyte. Other single analytes that can be measured by the method of the present invention include a dye, a therapeutic agent, and biological molecules such as a nucleotide, a nucleoside, and a neurotransmitter.

One aspect of the invention involves a method for determining the presence of a single analyte. A sample is provided comprising a plurality of analytes adsorbed on a plurality of aggregates. Each aggregate in the sample comprises a plurality of metal particles. The plurality of particles can also be referred to as a “cluster”. The aggregates and metal particles have particular dimensions that when exposed to electromagnetic radiation, an electromagnetic resonance is induced in the plurality of aggregates which in turn enhances an optical field surrounding a surface of the aggregate. Any emission from an analyte adsorbed on such an aggregate surrounded by an enhanced optical field is likewise enhanced. As used herein, “surface-enhancement” refers to the enhanced optical field and “surface-enhanced emission” as used herein refers to the enhanced emission from an analyte.

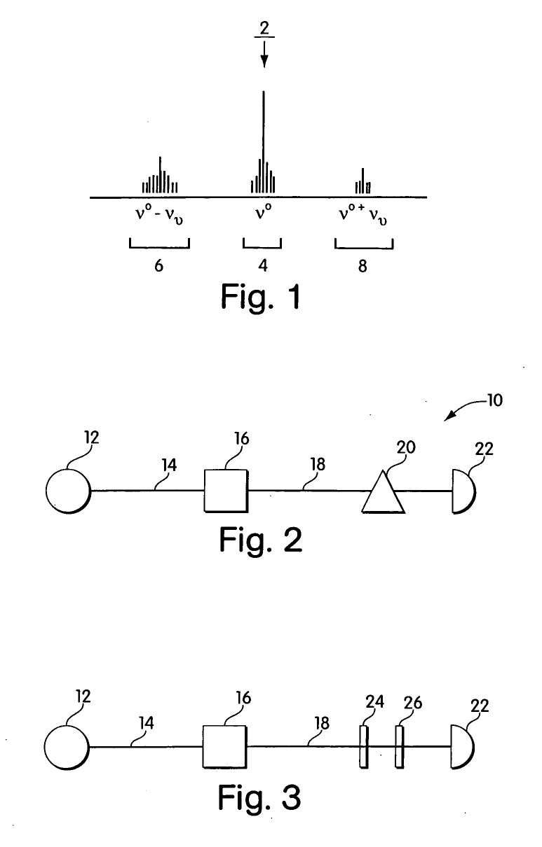

The sample is exposed to electromagnetic radiation to cause surface-enhanced emission, which can be surface-enhanced Raman emission and can be termed as “surface-enhanced Raman scattering” or SERS. “Spectral information” as used herein defines an emission spectrum, or a portion of an emission spectrum which can include a “spectral line” which refers to a single line of a spectrum. “Raman information” specifically refers to spectral information which comprises at least a portion of a Raman spectrum. FIG. 1 shows a schematic of a Raman spectrum 2. The Raman spectrum 2 consists of two sets of Raman signals termed “Stokes” lines and “anti-Stokes” lines. Referring to FIG. 1, a Raman spectrum contains one line due to photons involved in Rayleigh scattering 4, Stokes lines 6 in which photons have frequency values, v^{o}−v_{υ}, and anti-Stokes lines 8 in which photons have frequency values, v^{o}+v_{υ}. Resulting spectral information such as a Raman spectrum provides structural information on the plurality of analytes and a signal attributable to a lone analyte on an aggregate. In this technique and others a sample can be provided that contains aggregates adsorbing no analytes, aggregates adsorbing only one analyte and aggregates adsorbing more than one analyte. Preferably the sample includes mostly aggregates adsorbing no analytes and aggregates adsorbing only one analyte. Ideally, the sample is free of aggregates adsorbing more than one analyte. Aggregates can have a spherical or oval shape, or can be lined end to end to form a linear structure. The standard of measure for aggregates, known to those of ordinary skill in the art, is a mean diameter, or “dimension.”

As noted, the invention resides, in part, in the discovery that aggregate dimension can affect the ability to determine a single analyte via surface-enhanced emission spectroscopy. In this aspect, the present invention provides a plurality of aggregates adsorbing a plurality of analytes wherein at least one aggregate has a dimension of no more than about 200 nin and adsorbs only one analyte. Preferably, the at least one aggregate has a dimension of no more than about 175 nm, and more preferably no more than about 150 nm. In a particularly preferred embodiment the at least one aggregate that absorbs only one analyte has a dimension of between about 100 nm and 150 nm. In this set of embodiments preferably at least about 50% of the aggregates have a dimension no more than about 200 nm or other preferred dimensions above, more preferably at least about 70% of the aggregates have a dimension no more than about 200 nm or the above other dimensions.

In another set of embodiments the invention involves use of aggregates for surface-enhanced emission spectroscopy in which at least one aggregate of at least about 500 nm in dimension adsorbs a single analyte that is detected. This embodiment reflects the recognition of a technique for effective surface-enhanced emission spectroscopy using aggregates that are largely easily handleable. Aggregates that are of at least about 500 nm in dimension are much more easily handleable than are smaller aggregates, and thus in this embodiment about 500 nm is a critical lower range. The invention involves, in part, the recognition that easily handleable aggregates can be used with surface-enhanced emission spectroscopy when the sizes of particles that make up the aggregates are within preferred ranges described below. More preferably, the at least one aggregate is between about 500 nm and about 20 μm. More preferably, a sample is provided and subjected to surface-enhanced emission spectroscopy in which at least about 50% of the aggregates defining the sample are of a dimension greater than about 500 nm, more preferably at least about 70% of the aggregates of the sample have a dimension of greater than about 500 nm, more preferably still at least about 85% of the aggregates are of a dimension greater than about 500 nm. Other ranges embraced the invention includes samples in which the aggregate size ranges from about 500 nm to about 10 μm, or from about 500 nm to about 5 μm or 1 μm. Another aspect of the invention correlates the desired aggregate dimension with a number of particles in an aggregate. The invention provides a sample having a plurality of SERS-active aggregates comprising metal clusters of at least seven particles, preferably at least ten particles, more preferably at least twenty particles and more preferably still, at least thirty-five particles.

Aggregates and other surfaces identified according to the invention produce a very strong electromagnetic field enhancement due to resonance with the collective eigenmodes of the interacting particles in an aggregate of colloidal particles, to allow Raman detection of only one analyte having a surface that is a surface-enhanced Raman scattering (SERS-active) surface. The present invention provides a large Raman cross-section, resulting in a surface-enhanced Raman spectrum having an enhancement factor of at least 10¹⁰, where “enhancement factor” refers to the extent that surface-enhancement increases the intensity of Raman scattering. SERS-active surfaces are known, and are typically conducting surfaces having a high surface area with features capable of localizing a plasmon. The SERS-active surface may be a metal conducting surface selected from the group consisting of silver, gold, copper, lithium, sodium, potassium, indium, aluminum, platinum and rhodium. The aggregate comprises a plurality of particles having a surface that is an SERS-active surface. A surface of each particle may be a metal conducting surface selected from the group consisting of silver, gold, copper, lithium, sodium, potassium, indium, aluminum, platinum and rhodium surfaces.

One important aspect of the invention involves recognition that particle size of aggregate particles is important in obtaining Raman signals attributed to a single analyte. In this aspect the invention provides a sample including SERS-active aggregates including a plurality of particles in which at least one aggregate includes particles having a dimension of no more than about 100 nm, preferably no more than about 85 nm, more preferably no more than about 75 nm, more preferably no more than about 50 nm and in particularly preferred embodiment between about 10 nm to about 50 nm. Preferably, at least 50% of the aggregates in the sample are defined by particles of these sizes, more preferably at least about 70% of the aggregates of the sample are made of particles of these sizes. “Sample”, in the context of aggregates, defines aggregates of a single Raman experiment carrying immobilized analytes and exposed to Raman excitation.

A preferred set of embodiments includes all combinations of preferred aggregate sizes of particles that make up aggregates described above. For example, in one preferred embodiment at least 50% of the aggregates of a sample have a dimension of at least about 500 nm and are made up of particles of dimension of no more than about 100 nm. In another preferred embodiment at least 70% of aggregates of a sample have a dimension of between about 500 nm and 1 mm, and these aggregates are made of particles of dimension of between about 10 nm and about 50 nm.

The plurality of aggregates may be a colloidal suspension of aggregates dispersed in a medium such as water, an organic solvent or a gel. Colloidal suspensions are typically prepared by chemically reducing metal salts with reductants such as sodium borohydride and sodium citrate in aqueous or organic solutions. Colloidal suspensions can also be prepared by laser ablation of a solid metal. The plurality of aggregates may comprise clusters of particles deposited on a surface, and the clusters are referred to as “island films” in this embodiment. Metal clusters may be deposited on an electrode or on a substrate such as glass or quartz. The aggregates may be lithography-produced aggregates.

It is a further advantage of the present invention that a single analyte adsorbed on an aggregate has reduced Brownian motion compared to that of a single analyte dissolved in solution. Single molecule detection of analytes dissolved in solution are known in the art. When detecting analytes in solution, whether the analyte is adsorbed or not adsorbed on a surface, the analyte that is measured is located within a probed volume. The greater weight of an analyte adsorbed on an aggregate, however, results in a decreased Brownian motion compared to a single analyte dissolved in solution and thus a longer residence time within the probed volume, allowing an increase in intensity of a resulting signal.

Aggregates of the invention can be supplied as metal aggregates and combined with analytes for surface adsorption according to known methods, or analytes can be combined with aggregate material precursor that is formed into aggregates in situ, followed by analyte adsorption formation of aggregates according to preferred ranges described herein, from metal precursor material, can be carried out by those of ordinary skill in the art using known techniques. Formation can occur via the same irradiation that causes surface-enhanced excitation. For example, silver halide can be provided in solution or on a surface, combined with analyte, and exposed to laser radiation that causes both silver aggregate formation and surface-enhanced Raman excitation resulting in detection of a single analyte on an aggregate.

As noted, it is a feature of the invention that a variety of conditions are identified that allow single analyte determination using spectral information from surface-enhanced emission. To obtain a spectrum that has features dominated by a single analyte absorbed on an aggregate from a sample including many aggregates defining a colloidal metal solution, where each aggregate is a cluster of metal particles, a dilute analyte solution should be used to prepare the sample. The probability of adsorbing no more than a single analyte on each aggregate is increased if the colloidal metal solution is combined with a very dilute solution of analyte. This can result in a majority of a sample including aggregates absorbing no analytes and adsorbing only one analyte. Those of ordinary skill in the art can select preparation solutions suitable for maximizing the percentage of aggregate particles that carry only one analyte.

Another aspect of the invention involves obtaining spectral information such as at least a portion of a Raman spectrum from a single analyte adsorbed on a rough metal film, and obtaining spectral information from a rough metal film of a particular set of preferred surface feature sizes. Rough metal films for Raman spectroscopy are generally known, and include a plurality of protrusions and voids defining a rough surface. The plurality of protrusions and voids can correspond to a two-dimensional metal grating. The rough metal film can be prepared by depositing a metal film on a rough substrate such as CaF₂ or alumina, SiO₂ or other fine particle surfaces. In this aspect of the invention, feature sizes (indentations and protrusions) of the metal film, either vertically or horizontally measured, correspond to preferred aggregate sizes described above. Such surfaces preferably are prepared by depositing aggregates on a metal film as described herein, in terms of preferred aggregate size ranges and preferred particle size ranges and numbers of particles that make up the aggregates, onto the metal film. Metal films prepared in this way provide the ability to determine a single analyte molecule at a surface, such as a fragment of DNA or RNA, from spectral information derived from surface-enhanced emission.

In preferred embodiments of the invention, spectral information such as at least a portion of a Raman spectrum attributable to a single analyte that is free of an emission-enhancing aid is obtained. As used herein, “emission-enhancing aid” defines a component that, when exposed to a particular frequency or frequency range of electromagnetic radiation that would excite the species of interest (analyte) somewhat, produces greater excitation, thus a greater signal, than the species would alone. “Emission-enhancing aid” as used herein excludes surfaces of the invention. Emission-enhancing aids are known, and a non-limiting exemplary list includes dyes, pigments, and other chromophores, radioactive labels, fluorescent tags, fluorescence-enhancing matrices, and the like. Where fluorescence spectroscopy is used, as would be known to those of ordinary skill in the art the analyte should be spaced from the aggregate by a spacer of appropriate dimension. The spacer can be a molecular chain. In fluorescence embodiments the analyte also preferably is free of an emission-enhancing aid.

Another aspect of the invention involves SERS Raman spectroscopy to determine the presence of at least one molecule from at least one anti-Stokes line. As discussed previously, due to the Boltzmann distribution, anti-Stokes lines have considerably smaller signal intensities than those of Stokes lines. Consequently, the background level is substantially less than that of the Stokes lines which presents a considerable advantage for using the anti-Stokes lines to detect a single molecule. In enhancing the Raman scattering upon exposure of the at least one molecule to electromagnetic radiation, the method of the present invention, involving aggregates of particles having dimensions defined previously, allows enhancement of the intensity of anti-Stokes lines with respect to the background signal. Even a single molecule can be detected from at least one anti-Stokes line and Raman spectral information on the vibrational structure can be obtained from the anti-Stokes lines.

Electromagnetic radiation used in techniques and systems of the invention can be resonant or non-resonant. Resonant radiation corresponds to an energy capable of promoting a molecule to an excited state. Non-resonant radiation does not correspond to any electronic transitions of a molecule. Radiation that causes surface-enhanced emission such as Raman scattering can be resonant or non-resonant. Only a small portion of the energy supplied by the radiation is stored as vibrational energy by the molecule, and this vibrational energy can produce spectral information such as a Raman signal.

In the present invention, the electromagnetic radiation is preferably non-resonant and more preferably near infrared radiation. “Near infrared radiation” refers to the portion of electromagnetic radiation having energy values intermediate those of visible radiation and far infrared radiation. Non-resonant radiation has not been used, to the applicants' knowledge, for detection and analysis of a single analyte and this possibility is provided by aggregate and surface feature sizes, and particle sizes making up aggregates, of the invention. Another aspect the invention involves exposing a surface, on which is absorbed a single analyte, to non-resonant radiation and obtaining spectral information such as a Raman spectrum including a signal attributable to the only one analyte.

In another embodiment a method for general field enhancement is provided. As discussed previously, exposing a plurality of aggregates and metal to electromagnetic radiation induces an electromagnetic resonance in the plurality of aggregates to cause an enhanced optical field and enhanced emission of analytes adsorbed on the plurality of aggregates. “General field enhancement”, as used herein refers to the enhancement of the optical field. The present invention allows the enhancement to be increased considerably when a plurality of aggregates having dimensions defined as above is exposed to near infrared radiation. For example, when the emission is Raman emission, a surface-enhanced Raman spectrum resulting from the general field enhancement experiences an enhancement factor of at least 10¹⁰.

Because non-resonant radiation does not correspond to electronic transitions, an advantage of exposing a molecule to non-resonant radiation is that photobleaching is avoided. “Photobleaching” is defined herein as exposing a molecule to radiation such that the molecule is promoted to an excited electronic state which results in changing the electronic structure of the molecule such that a chemical change occurs. The chemical change may result in a change in molecular structure or even destruction of the molecule, and consequently spectral information such as a Raman signal attributed to the molecule may undergo a frequency shift, a decrease in intensity or disappear. It is a feature of the invention that exposing the analyte to non-resonant radiation prevents chemical changes due to electronic structural changes from occurring, and preventing photobleaching.

In another aspect the invention provides a technique for obtaining a unique piece of spectral information, such as a unique portion of a spectrum defining a single line, attributed to a single DNA or RNA fragment which can be any portion of a DNA or RNA strand comprising at least one base. In this technique, a single fragment of DNA or RNA is adsorbed onto a rough aggregate-bearing metal surface or a plurality of aggregates. Unlike prior art techniques, identification of the single fragment in the present invention does not require the use of an emission-enhancing aid such as a dye. The present invention involves successfully obtaining surface-enhanced emission spectral information, such as Raman identification of individual DNA or RNA fragments because of the aggregate size and/or particle size defining aggregates of the invention as defined or surface feature sizes of a rough metal film, as defined.

In another embodiment, a method for sequencing at least a portion of DNA or RNA is provided. DNA or RNA is cleaved into fragments and each fragment is allowed to become individually adsorbed onto a rough aggregate-bearing metal surface or a plurality of aggregates. When each fragment is immobilized individually onto aggregates, this technique presents advantages over prior art technique such as that described in U.S. Pat. No. 4,962,037 (Jett, et al.). The technique of Jett, et al., requires cleavage of the individual bases from fragments in which the bases have been tagged with a characteristic fluorescent dye. Jett discloses cleaving the individual bases into a solution. As discussed previously, one advantage of a technique of the invention analyzing surface-adsorbed individual analytes as opposed to nonsurface-adsorbed individual analytes in a liquid is that the Brownian motion of surface-adsorbed analytes is decreased considerably, allowing the analyte to have a longer residence time in the probed volume than nonsurface-adsorbed analytes. In one embodiment, DNA or RNA is cleaved with nucleases known in the art and each resulting fragment is allowed to become surface-adsorbed on a plurality of aggregates in a liquid stream.

In another embodiment, single fragments of DNA or RNA are allowed to become surface-adsorbed on a rough metal surface, where each fragment comprises at least one base. A spectroscopic determination can readily be made as to the identity of an individual fragment by identifying its spectral information relative to location on the surface. In yet another embodiment the portion of a DNA or RNA is cleaved and the resulting fragments allowed to become surface-adsorbed on a rough metal surface. The method involves cleaving the DNA or RNA portion and applying the resulting individual fragments to a moving metal surface to sequentially apply the individual fragments to the surface. This can be carried out by cleaving DNA or RNA, using nucleases as known, and spreading the individual fragments that result on the surface. If the DNA or RNA portion is cleaved with exonucleases, droplets containing a single fragment cleaved in this manner can be provided on a surface and different fragments can be provided at different locations by moving the surface relative to the source of the droplets. This can result in individual fragments being located at different readily determinable locations on a metal film. For example, if the metal film is moving at a speed proportional to the speed of DNA or RNA cleavage and application to the film, determination can readily be made as to the identity to the individual fragments by identifying their spectral information relative to locations on the surface.

In another aspect the invention provides a method of carrying out spectroscopy using surface-enhanced emission to obtain spectral information in a selected electromagnetic radiation wavelength range without use of spectrograph. Typically, a Raman spectrometer includes an optical excitation system which produces electromagnetic radiation in a first wavelength range in the absence of a spectrograph or other prior art wavelength selection systems relating to Raman spectrometers. To shift the wavelength range, the prior art teaches the use of a spectrograph which allows the Raman spectrometer to produce electromagnetic radiation in a second wavelength range. In this aspect of the invention, the wavelength range can be shifted in the absence of a spectrograph by the use of at least one filter, or two filters, typically selected from a hi-pass filter and low-pass filter, to define a wavelength range of spectral information for analysis. One or more filters can be provided in front of a detector of a Raman system. In a preferred embodiment, when the Raman spectrum is shifted, the second range is narrower, “narrower” being defined as the second range corresponding to a spectrum which is a portion of a surface-enhanced emission spectrum and can define a single signal such as a single Raman line (band). The advantage of a spectrograph-free Raman system as compared with a spectrograph-based system is higher throughput and light efficiency.

FIG. 2 represents a prior art surface-enhanced spectral system such as a surface-enhanced Raman spectroscopy arrangement 10. System 10 includes a source 12 of electromagnetic radiation (a laser). Excitation radiation 14 generated by source 12 interacts with a sample 16. Sample 16 can include a plurality of analytes adsorbed on a plurality of aggregates, or on a rough surface, as defined herein. Surface-enhanced emission 18 passes through a spectrograph 20 which prepares the emission for detection by detector 22.

FIG. 3 illustrates a system of the invention that is similar to that of FIG. 2, but without spectrograph 20. Instead, at least one filter 24, and more commonly two filters 24 and 26 at least, are provided to produce spectral information via detector 22 defining a portion of a Raman spectrum, for example. The portion is less than a complete Raman spectrum, and can be less than 5 Raman lines, and in another embodiment, less than two Raman lines, or a single Raman line. In one example, filter 24 is a high-pass filter and filter 26 is a low-pass filter that together isolate a wavelength range that allows only a single Raman line to pass. This system provides a higher throughput and efficiency compared to the prior art system.

The function and advantage of these and other embodiments of the present invention will be more fully understood from the examples below. The following examples are intended to illustrate the benefits of the present invention, but do not exemplify the full scope of the invention.

### EXAMPLE 1

### Detection of a Single Molecule of Crystal Violet

This example illustrates the ability to detect a single molecule of a dye, specifically crystal violet. Colloidal solutions were prepared by a standard citrate reduction procedure (J. Phys. Chem. 1982, 86, 3391). A 10⁻² M NaCl solution was added to achieve optimum SERS-enhancement factors. Electron micrographs of the solution taken before the addition of the targeted compound are shown in Kneipp et al., Laser Scattering Spectroscopy of Biological Objects, Studies in Physics and Theoretical Chemistry, Vol.45 p.451 (Elsevier, 1987). The resulting colloidal solution is slightly aggregated and consists of small 100-150 nm sized clusters (aggregates). The solution extinction spectrum shows a maximum at about 425 nm. The probed volume is 30 pL.

Samples were prepared in a manner that maximized the percentage of aggregates carrying single analytes by adding 5×10⁻¹³ M crystal violet solution in methanol to this colloidal solution in a volume ratio of 1:15, resulting in a final sample concentration of 3.3×10⁻¹⁴ M, resulting in an average of 0.6 molecules in the probed 30 pL volume. From the total silver in the colloidal solution the number of individual silver clusters in the probed volume was estimated to be about 100. The ratio of the number of dye molecules to the number of silver cluster was ˜0.6:100. Repeated checking of the extinction spectra of the sample solution during and after SERS measurement time showed no change implying no further aggregation after the addition of crystal violet.

The excitation source was an argon-ion laser pumped cw Ti:sapphire laser operating at 830 nm with a power of about 200 mW at the sample. Dispersion was achieved using a Chromex spectrograph with a deep depletion CCD detector. A water immersion microscope objective (×63, NA 0.9) was brought into direct contact with a 30 μl droplet of sample solution for both excitation and collection of the scattered light. The probed volume was estimated to be approximately 30 pL. The average residence time of a particle in the probed volume can be roughly estimated to be between 10 and 20 seconds, which is at least ten times longer than the measurement time.

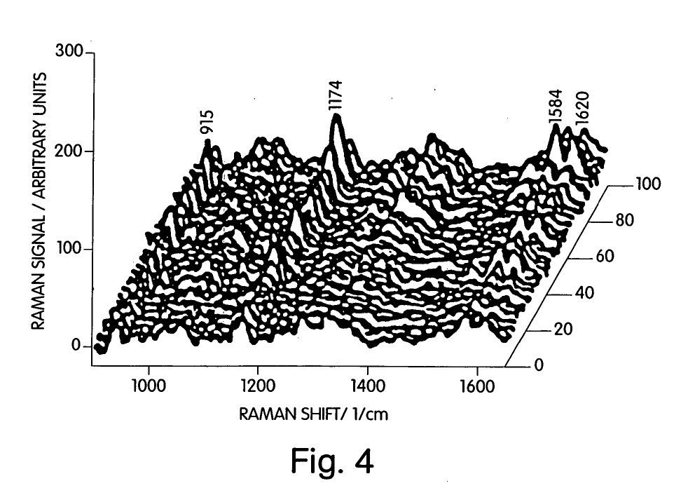

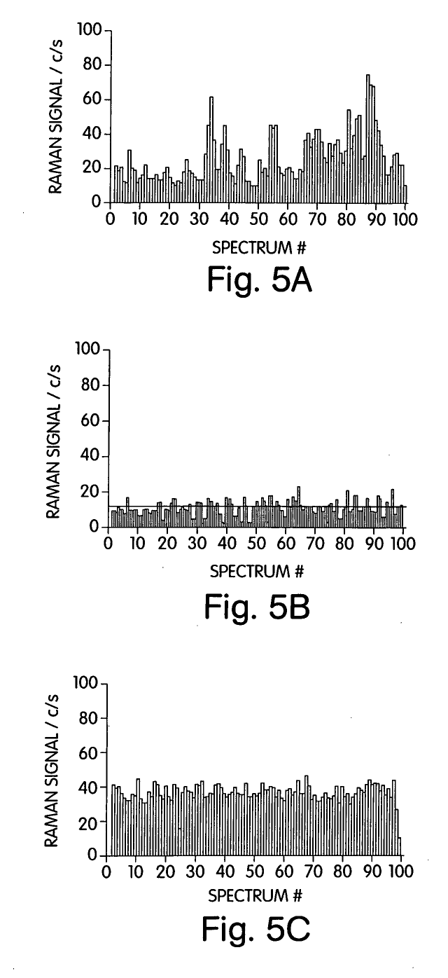

FIG. 4 shows 100 surface-enhanced Raman scattering (SERS) spectra measured in time sequence from a probed volume which contains an average of 0.6 crystal violet molecules. FIG. 5(a) displays the peak heights of the 1174 cm⁻¹ line for the 100 SERS spectra. Spectra 1 to about 30 show no significant peak intensity, indicating that these measurements were taken when the analyte was not present in the probed volume. Within spectra 30 and 40, the crystal violet molecule diffuses into the probed volume as evidenced by the appearance of several peaks having significant intensity. Measurements of a control solution of colloidal solution with no dye present are shown in FIG. 5(b) which illustrates the background level. The horizontal line at 14 counts/s is the mean background signal. The threshold for signal detection is set to 25 counts/s which is three times the standard variation in the mean background signal. FIG. 5(a) shows that about 40 signals measured in the presence of dye molecules meet this criterion.

For comparison, FIG. 5(c) shows an analogous measurement for the 1030 cm⁻¹ Raman line of 3 M methanol in colloidal silver solution (about 1014 molecules of methanol in the probed volume). The methanol concentration is adjusted to achieve approximately the same count rate for “many” molecules as for a single crystal violet molecule in order to compare statistics at approximately the same signal-to-noise levels. Previous experimental data showed no indication of any SERS enhancement of the methanol Raman signal. Since there are about 1014 times more molecules of methanol than of crystal violet in the probed volume, the same signal strengths for the methanol Raman line and for the crystal violet SERS line confirm an enhancement factor of about 10¹⁴ and cross sections on the order of 10⁻¹⁷ to 10⁻¹⁶ cm²/molecule.

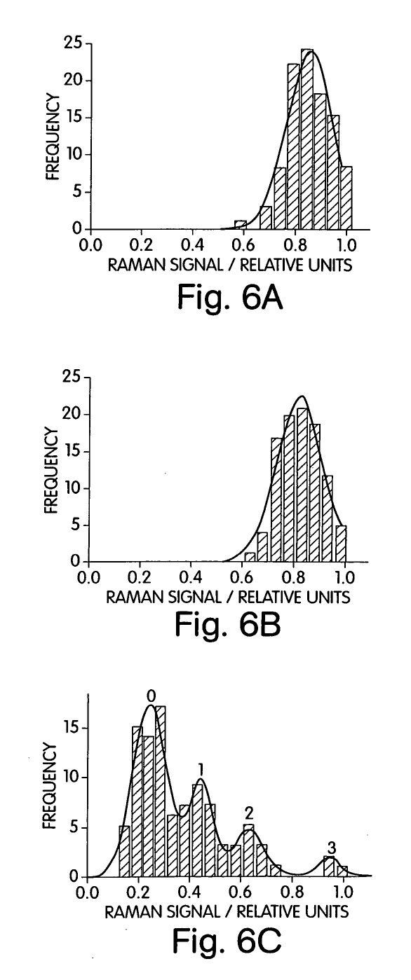

FIG. 6 presents a statistical analysis of the Raman signals measured in time sequence using 20 bins whose widths are 5% of the maximum of the observed signals (x axis). The y axis displays the frequency of the appearance of the appropriate signal levels of the bin. FIG. 6(a) gives the statistical analysis of 100 normal Raman measurements of 10¹⁴ methanol molecules in the probed volume. As expected, the Raman signal of many methanol molecules shows a Gaussian statistical distribution. FIG. 3(c) displays statistical analysis of 100 SERS measurements (signal of the 1174 cm⁻¹ Raman line) of 0.6 crystal violet molecules in the probed volume. In contrast to the Raman signal of many molecules, the statistical distribution of the “0.6 molecules SERS signal” exhibits four relative maxima which are reasonably fit by the superposition of four Gaussian curves whose areas are roughly consistent with a Poisson distribution for an average number of 0.5 molecule. This reflects the probability to find 0, 1, 2 or 3 molecules in the probed volume during the actual measurement. Comparing the Poisson fit with the 0.6 molecule concentration/volume estimate we conclude that about 80% of molecules are adsorbed.

FIG. 6(b) shows that the characteristic Poisson distribution vanishes and the statistics of the SERS signal becomes more Gaussian if we increase dye concentration by a factor of 10.

### EXAMPLE 2

### 1,1′-diethyl-2,2′-cyanine (pseudoisocyanine)

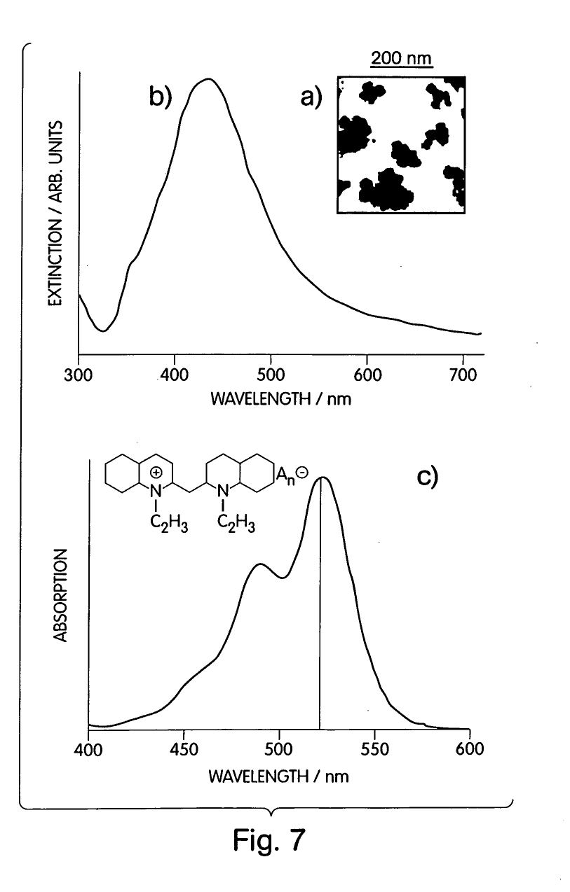

This example illustrates the detection of a single molecule of pseudoisocyanine. A colloidal solution was prepared by a standard citrate reduction procedure described in Lee, et al., J. Phys. Chem. 1982, 86, 3391. Sodium chloride was added in 10⁻² M concentration to achieve optimum SERS conditions. Sodium chloride in such low concentration does not change the colloidal structure as is demonstrated by the unchanged extinction spectra of the colloidal solution after additions of sodium chloride. A 10⁻¹² M pseudoisocyanine solution in methanol was added to this colloidal solution to produce pseudoisocyanine solutions having concentrations of 5×10⁻¹³ M and 3×10⁻¹³ M. The average number of molecules contributing to the Raman signal at these dye concentrations in a 3 pL probed volume was estimated to be 0.9 and 0.6, respectively. FIG. 7 shows an extinction spectrum of the colloidal solutions and electron micrographs of 100 nm-200 nm silver clusters which are SERS-active substrates. These clusters are formed from individual 15-40 nm silver colloids.

The excitation source was an argon-ion laser pumped cw Ti:sapphire laser operating at 830 nm with a power of about 100 mW at the sample. The absorption band of pseudoisocyanine at 520 nm is well separated from the 830 nm excitation wavelength. Dispersion was achieved using a Chromex spectrograph with a deep depletion CCD detector. A water immersion microscope objective (×63, NA 0.9) was brought into direct contact with a 30 μL droplet of sample solution for both excitation and collection of the scattered light. The probed volume was estimated to be approximately 3 pL. The average residence time of a particle in the probed volume can be roughly estimated to be between 3 and 5 seconds.

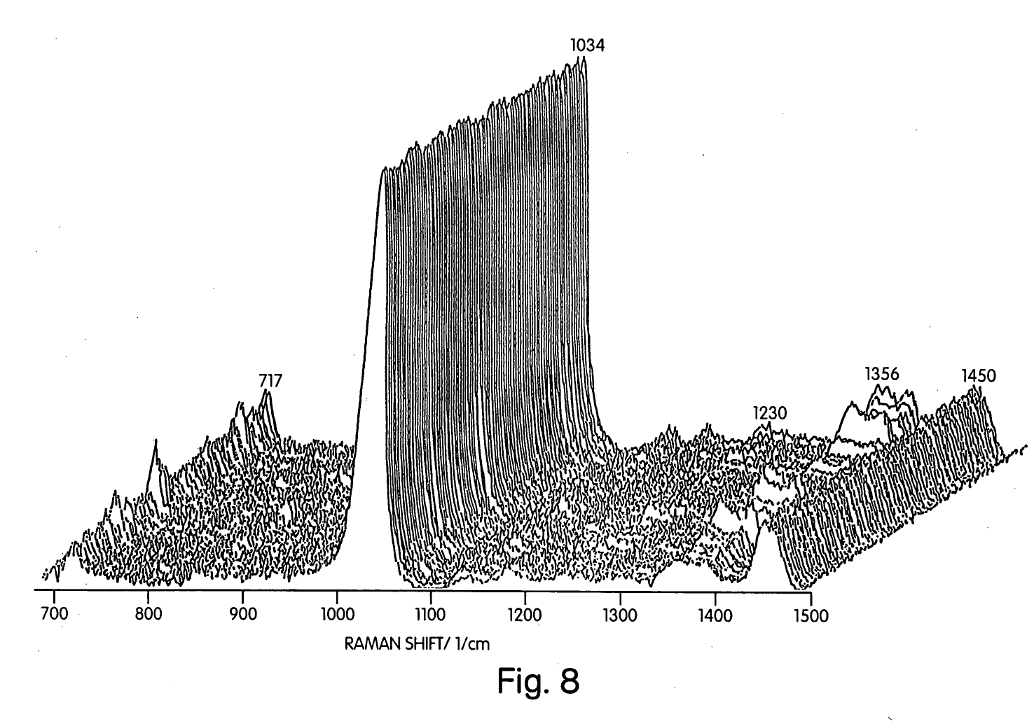

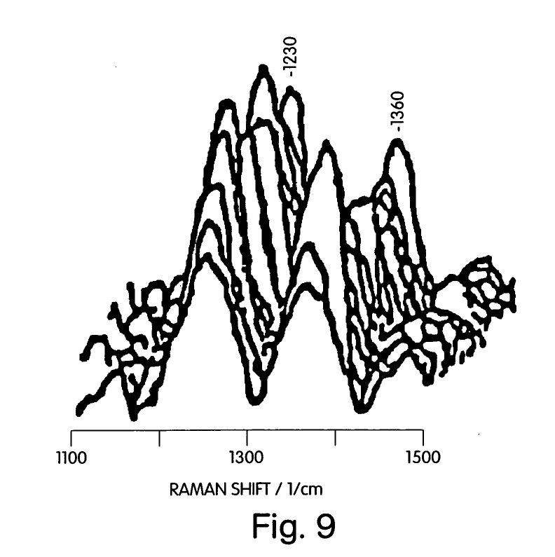

FIG. 8 shows typical Raman spectra measured in one second collection time from a sample which contains 0.9 pseudoisocyanine molecules and about 10¹³ methanol molecules in the probed volume. Pseudoisocyanine SERS lines appear at 717 cm⁻¹, 850 cm⁻¹, 1230 cm⁻¹, and as a doublet at 1360 cm⁻¹. The Raman frequencies are in agreement with pseudoisocyanine SERS spectra reported at visible excitation. The relative intensities of the lines are slightly changed due to non-resonant near infrared excitation. Methanol does not show any SERS enhancement and gives rise to Raman lines at 1034 and at 1450 cm⁻¹.

FIG. 8 clearly demonstrates that pseudoisocyanine SERS lines and methanol Raman lines show different statistical behavior. Whereas the Raman lines of the 10¹³ methanol molecules appear at relatively uniform signal levels, strong fluctuations in the pseudoisocyanine SERS signals appear due to Brownian motion of the colloidal silver particles which carry single dye molecules into and out of the probed volume. During an actual measurement, just 0, 1, 2 or relatively unlikely, 3 pseudoisocyanine molecules contribute to the SERS spectrum resulting in different peak heights of the Raman lines.

FIG. 9 shows spectra measured from the same sample between 100 cm⁻¹ and 1500 cm⁻¹ Raman shift at the anti-Stokes side in 1 s collection time which demonstrates the ability to use the anti-Stokes lines to detect single molecules. Single molecule anti-Stokes Raman lines appear at 1360 and 1230 cm⁻¹ at about 20 times the lower signal level than Stokes lines. Methanol anti-Stokes signals at 1450 are not detected under these experimental conditions.

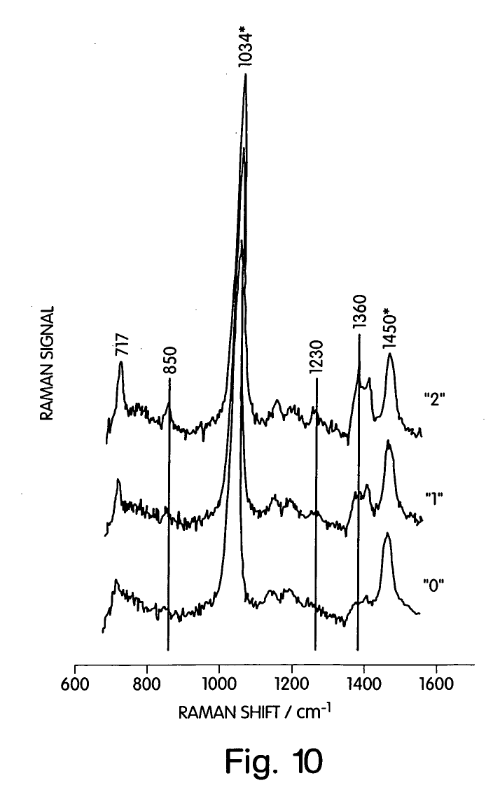

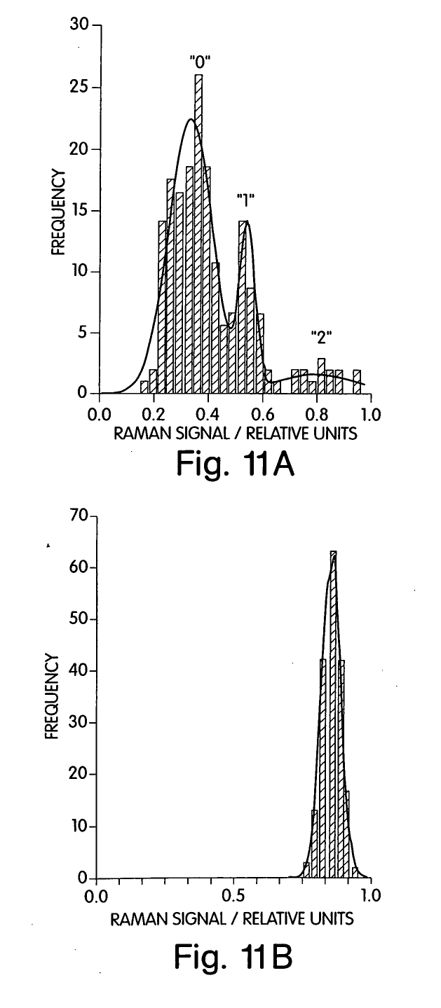

FIG. 10 displays three typical Raman spectra measured from a sample which contains an average of 0.5 pseudoisocyanine molecules and 10¹³ methanol molecules (3×10⁻¹³ M pseudoisocyanine and 6 M methanol in 3 pL probed volume). Traces show typical spectra as statistically appear and represent about 1, 2 or 0 pseudoisocyanine molecules in the probed volume.

FIG. 11 shows the results of a statistical analysis of the pseudoisocyanine SERS signal at 1360 cm⁻¹ (FIG. 11(a)) and of the methanol Raman signal at 1450 cm⁻¹ (FIG. 11(b)). The scattering signals of 200 measurements were divided into 30 bins (x-axis). The y-axis displays the frequency of the appearance of the appropriate signal levels of the bin. As expected, the Raman signal of 10¹³ methanol molecules shows a Gaussian statistical distribution (FIG. 11(b)). In contrast, the statistical distribution of the “0.5 pseudoisocyanine molecules SERS signal” can be reasonably fit by the superposition of three Gaussian curves whose areas are roughly consistent with a Poisson distribution for an average number of 0.4 molecules. This reflects the probability to find 0, 1 or 2 molecules in the scattering volume during the actual measurement. Comparing the 0.4 molecule fit with the 0.5 molecule concentration/volume estimate we conclude that about 80% of the pseudoisocyanine molecules were detected by SERS.

The change in the statistical distribution of the Raman signal from Gaussian to Poisson when the average number of dye molecules in the scattering volume is one or less is evidence for single molecule detection by SERS.

As FIGS. 9 and 10 demonstrate, single molecule spectra can be measured at a signal to noise ratios of about 10 in a 1 second collection time for about 100 mW excitation focused to about 3×10⁻⁷ cm². Assuming a SERS cross section on the order of 10⁻¹⁷-10⁻¹⁶ cm²/molecule and a vibrational lifetime on the order of 10 picoseconds/18/, saturation of SERS will be achieved at 10⁸-10⁹ W/cm² excitation intensity. Applying the same Raman system (same focusing condition of the excitation laser, same signal collection and detection efficiency), under saturation conditions, the collection time for single molecule spectra could be reduced by a factor 1000 and we should be able to measure single molecule SERS spectra in milliseconds.

### EXAMPLE 3

### Crystal Violet on Silver Particles and Colloidal Aggregates

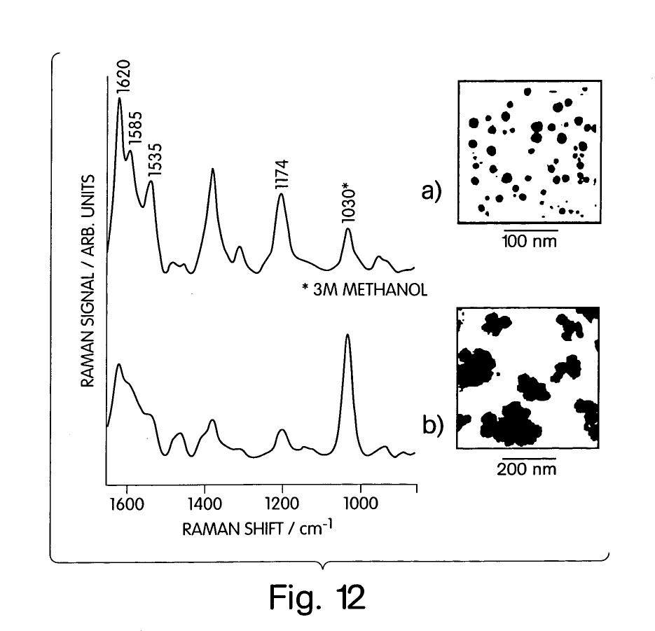

In this example, the SERS enhancement factors are compared for crystal violet (CV) adsorbed on spatially isolated 10-25 nm sized spherical colloidal silver particles and on colloidal aggregates of various sizes between 100 nm and 20 μm. Colloidal solutions were prepared by a standard citrate reduction procedure (Lee, et al., as in Example 1), or by laser ablation (Fojtik, et al., Ber. Bunsenges, Phys. Chem. 97 (1993) 252; Nedderson, et al., Appl. Spectry 47 (1993) 1959). Experiments are performed at 407 nm excitation (single particle plasmon resonance) and at 830 nm NIR excitation. From the absorption spectrum of crystal violet, it can be concluded that at these wavelengths nearly no molecular resonance Raman effect contributes to the observed total enhancement. The colloidal solutions have been prepared by a standard citrate reduction procedure or by laser ablation. SERS samples are prepared as described in Example 1. When small droplets of sample solution are dried on a microscope cover slide, dye loaded silver clusters of various sizes are fixed on the glass slide and the excitation laser can be focused (spot size approximately 3 μm) onto desired μm-clusters or onto areas between them which are covered with 100-500 nm (submicroscopic) silver clusters. FIG. 12 compares SERS at 407 nm excitation for crystal violet on isolated small spheres (FIG. 12(a)) and on small colloidal clusters (FIG. 12(b)). SERS enhancement is estimated by comparing the signal strength of the 1174 cm⁻¹ CV SERS band and the 1030 cm⁻¹ methanol Raman band and by taking into account the different concentrations of both molecules to be on the order of 10⁶ for spatially isolated small colloids and 10⁷-10⁸ for colloidal clusters. Since ablating silver in distilled and deionized water made the isolated small colloids, no special “chemical activation” (except silver ions) should exist. The value 10⁶ is in agreement with electrostatic estimates of enhancement factors for isolated spherical silver particles. Thus for visible radiation, the enhancement shown in colloidal clusters is greater than for isolated spherical silver particles.

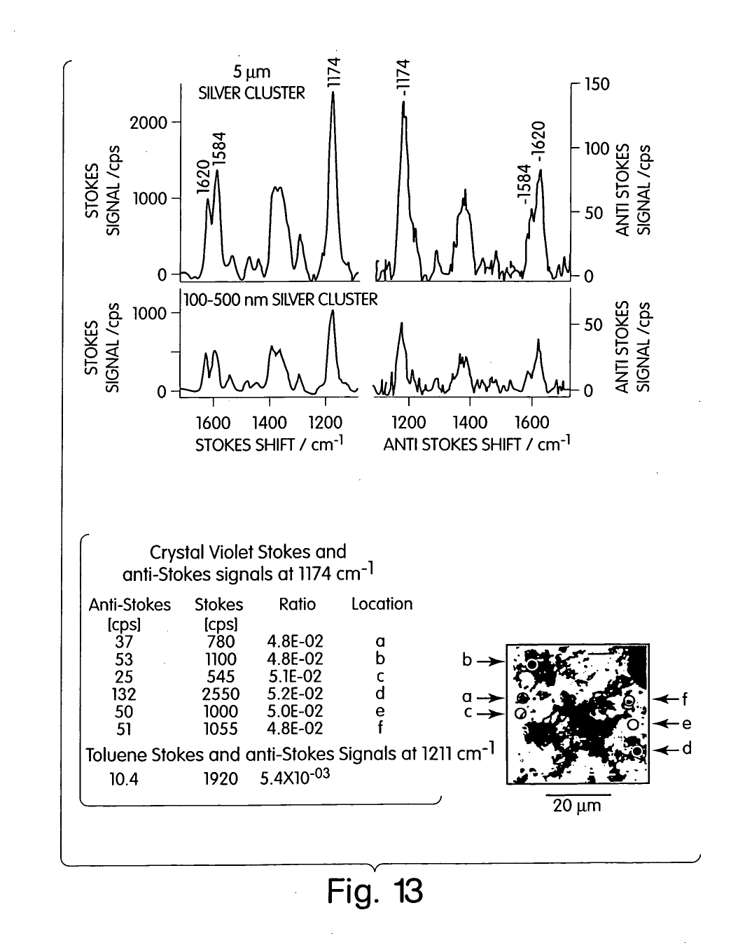

The enhancement factor for colloidal clusters versus isolated particles is considerably increased when the sample is exposed to near infrared radiation excitation. No SERS signal is measured for molecules on small isolated spheres at near infrared 830 nm excitation, due to the absence of single plasmon resonance at this wavelength. In contrasts enhancement factors for colloidal clusters at near infrared excitation increase tremendously and can be estimated from the obtained pumping of molecules to the first excited vibrational state due to the strong Raman process. FIG. 13 displays two sets of Stokes and anti-Stokes spectra and gives anti-Stokes to Stokes ratios measured from crystal violet on clusters of different sizes. Ratios between anti-Stokes and Stokes SERS signals from various clusters, which are constant within the accuracy of our measurement give an experimental proof of scaling invariance of the enhancement and these experiments provide a strong argument for an electromagnetic field enhancement related to colloidal cluster i.e. the enhancement factor is independent of cluster size. From the experimentally observed pumping, SERS cross sections of ˜10¹⁶ cm²/molecule or enhancement factors on the order of 10¹⁴ can be inferred in agreement with previous results for crystal violet. The increase of about 6 to 7 orders of magnitude for SERS enhancement on colloidal silver clusters when the excitation wavelength is shifted from 407 nm to 830 nm is in relatively good agreement with theoretical estimates. Phys. Rev. B, B46, 2821 (1992).

### EXAMPLE 4

### Adenosine monophosphate (AMP) and Adenine

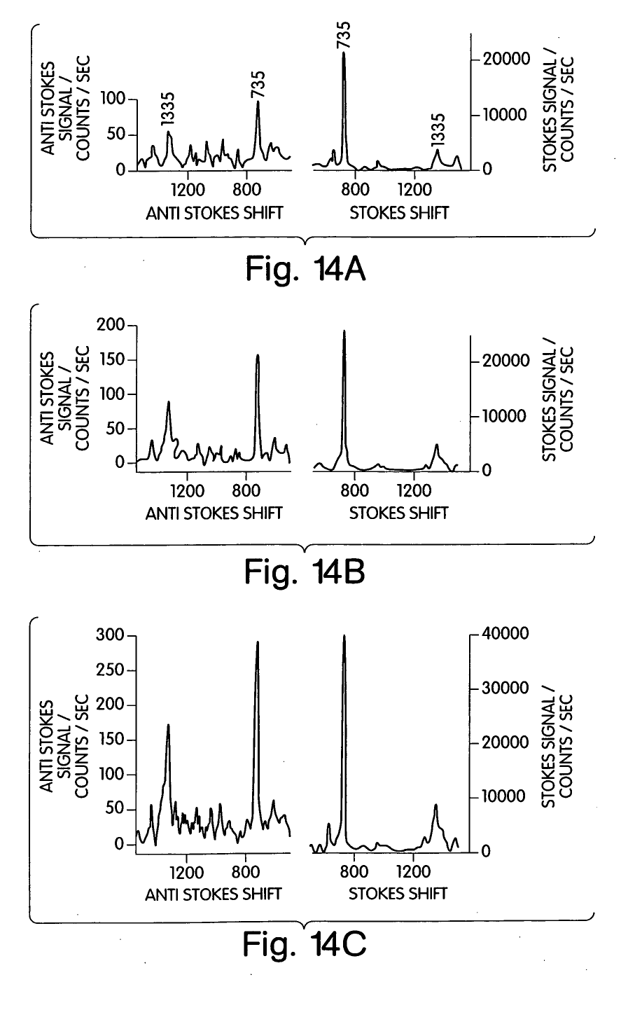

The ability to detect adenosine monophosphate and adenine provide an example for applying the methods of the present invention to DNA or RNA base sequencing. Colloidal solutions were prepared by a standard citrate reduction procedure (Lee, et al., as in Example 1), or by laser ablation (Fojtik, et al., Ber. Bunsenges, Phys. Chem. 97 (1993) 252; Nedderson, et al., Appl. Spectry 47 (1993) 1959). Experimental conditions described in Example 3 for near infrared excitation are also used here. FIG. 14 shows surface enhanced Stokes and anti-Stokes Raman spectra of adenosine monophosphate (AMP) and of adenine. Spectra display the strong Raman line of the adenine ring breathing mode at 735 cm⁻¹ and lines in the 1330 cm⁻¹ region. SERS spectra of adenine and AMP are identical showing sugar and phosphate does not prevent the strong SERS effect of adenine.

Effective Raman cross-sections of the order of 10⁻¹⁶ cm²/molecule can be inferred from the observed anti-Stokes to Stokes signal ratio. A comparison between anti-Stokes and Stokes adenine spectra measured from clusters of various sizes between about 100 nm and 10 μm (for example compare FIG. 14(b) and 14(c)) confirms the independence of the SERS enhancement factor or cluster size for adenine.

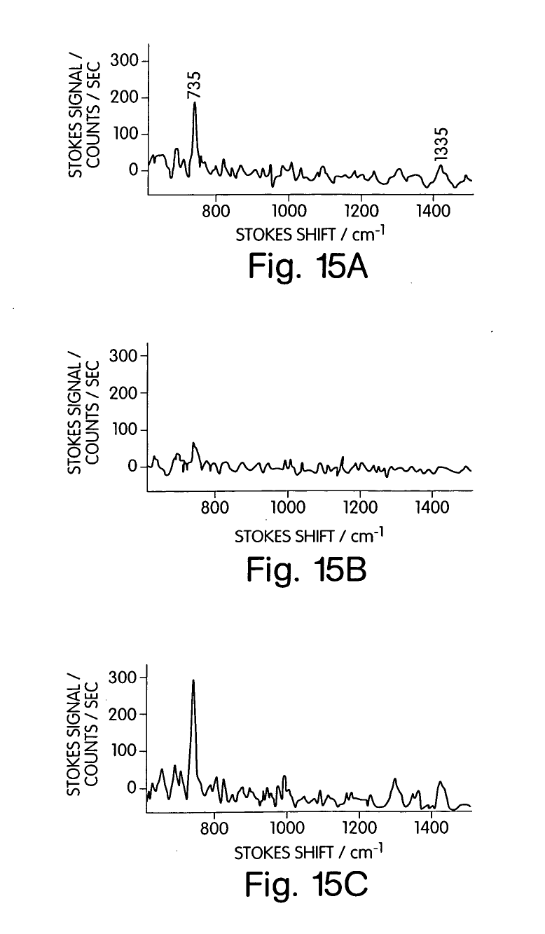

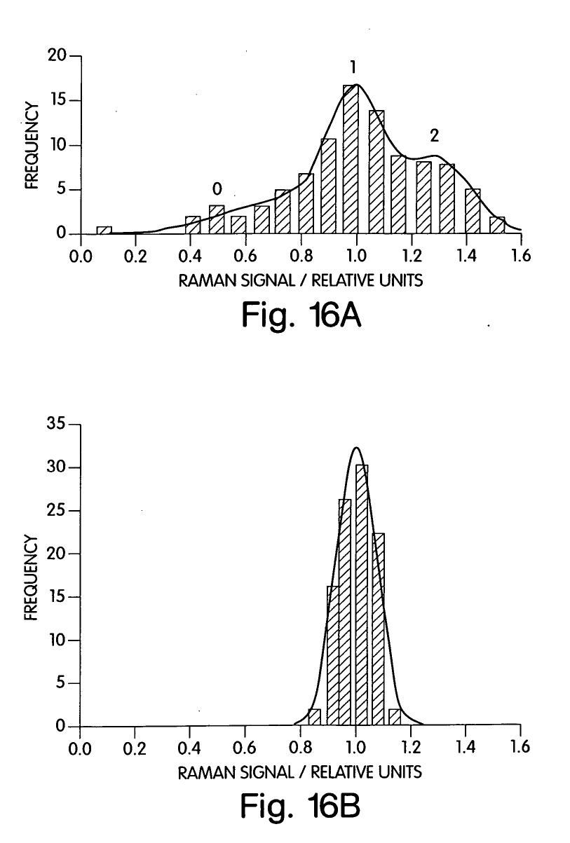

FIG. 15 represents selected typical spectra collected in 1 second from samples which contain an average of 1.8 adenine molecules in a probed 100-fl volume. The drastic changes disappear for 10 times higher adenine concentration when the number of molecules in the probed volume remains statistically constant. FIG. 16 gives the statistical analysis of adenine SERS-signals (100 measurements) from 18 molecules and from an average of 1.8 molecules in the probed 100-fl volume. The change in the statistical distribution of the Raman signal from Gaussian (FIG. 16(b)) to Poisson (FIG. 16(a)) reflects the probability to find 0, 1, 2 (or 3) molecules in the probed volume during the actual measurement and is evidence that single molecule detection of adenine by SERS is achieved. Comparing the 1.3 molecule fit with the 1.8 molecule concentration/volume estimate we conclude that 70-75% of the adenine molecules were detected by SERS.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described.

1. A method for determining the presence of at least one analyte, comprising:
providing a sample comprising a plurality of aggregates of size of at least about 500 nm adsorbing a plurality of analytes;
exposing the sample to electromagnetic radiation to cause surface-enhanced emission;
obtaining spectral information of the sample, wherein at least one spectral line of the information represents a single analyte adsorbed on one of the plurality of aggregates; and
determining the presence of the single analyte from the at least one spectral line.

2. A method as in claim 1, the exposing step involving exposing the sample to electromagnetic radiation and causing Raman scattering of the sample, and the obtaining step comprising obtaining Raman information of the sample, wherein a single Raman line of the information represents the single analyte.

3. A method as in claim 1, wherein the sample is free of an emission-enhancing aid.

4. A method as in claim 1, wherein the spectral information is a surface-enhanced Raman spectrum, having an enhancement factor of at least about 1010.

5. A method as in claim 1, wherein each aggregate of the plurality of aggregates comprises a plurality of metal particles.

6. A method as in claim 5, wherein the plurality of metal particles is selected from the group consisting of silver, gold and copper particles.

7. A method as in claim 6, wherein the aggregate is formed in situ by exposure to the electromagnetic radiation.

8. A method as in claim 1, wherein the plurality of aggregates is selected from the group consisting of colloids suspended in a medium, aggregates deposited on a substrate and lithography-produced metal aggregates.

9. A method as in claim 8, wherein the medium is selected from the group consisting of water, an organic solvent and a gel.

10. A method as in claim 8, wherein the substrate is selected from the group consisting of an electrode, a glass layer and a quartz layer.

11. A method as in claim 1, wherein the sample consists essentially of a plurality of aggregates of from about 500 nm to about 20 microns in dimension.

12. A method as in claim 1, wherein the electromagnetic radiation is non-resonant radiation.

13. A method as in claim 12, wherein the electromagnetic radiation is near infrared radiation.

14. A method as in claim 1, wherein the spectral information is Raman information that defines less than a complete Raman spectrum.

15. A method as in claim 14, wherein the spectral information is less than 5 Raman lines.

16. A method as in claim 14, wherein the spectral information is less than 2 Raman lines.

17. A method as in claim 1, wherein the spectral information is a single Raman line.

18. A method as in claim 1, wherein the single analyte is a dye.

19. A method as in claim 1, wherein the single analyte is selected from the group consisting of thymine, adenine, cytosine, guanine, and uracil.

20. A method as in claim 1, wherein the single analyte is selected from the group consisting of nucleotides and nucleosides.

21. A method as in claim 1, wherein the single analyte is a therapeutic agent.

22. A method as in claim 1, wherein the single analyte is a neurotransmitter.

23. A method for determining the presence of an analyte, comprising:
providing a sample comprising a plurality of aggregates adsorbing a plurality of analytes, wherein at least one aggregate of the plurality of aggregates comprises a metal cluster of at least seven particles and adsorbs only one analyte;
exposing the sample to electromagnetic radiation to cause surface-enhanced emission;
obtaining spectral information of the sample, wherein the only one analyte contributes to the spectral information; and
determining the presence of the only one analyte from the spectral information.

24. A method as in claim 23, the exposing step involving exposing the sample to electromagnetic radiation to cause Raman scattering, and the obtaining step involves obtaining a Raman spectrum of the sample, wherein the only one analyte contributes to at least one Raman signal of the Raman spectrum.

25. A method as in claim 23, wherein the plurality of aggregates comprises a metal cluster of at least ten particles.

26. A method as in claim 23, wherein the plurality of aggregates comprises a metal cluster of at least twenty particles.

27. A method as in claim 23, wherein the plurality of aggregates comprises a metal cluster of at least thirty-five particles.

28. A method as in claim 23, wherein the sample is free of an emission-enhancing aid.

29. A method as in claim 23, wherein the Raman spectrum is a surface-enhanced Raman spectrum, having an enhancement factor of at least 1010.

30. A method as in claim 23, wherein the metal particles are selected from the group consisting of silver, gold and copper particles.

31. A method as in claim 23, wherein the aggregate is formed in situ by exposure to the electromagnetic radiation.

32. A method as in claim 23, wherein the plurality of aggregates is selected from the group consisting of a colloids suspended in a medium, aggregates deposited on a substrate and lithography produced metal aggregates.

33. A method as in claim 32, wherein the medium is selected from the group consisting of water, an organic solvent and a gel.

34. A method as in claim 32, wherein the substrate is selected from the group consisting of an electrode, a glass layer and a quartz layer.

35. A method as in claim 23, wherein the at least one aggregate has a dimension of at least about 500 nm.

36. A method as in claim 23, wherein the electromagnetic radiation is non-resonant radiation.

37. A method as in claim 36, wherein the electromagnetic radiation is near infrared radiation.

38. A method as in claim 23, wherein the single analyte is a dye.

39. A method as in claim 23, wherein the single analyte is selected from the group consisting of thymine, adenine, cytosine, guanine, and uracil.

40. A method as in claim 23, wherein the single analyte is selected from the group consisting of nucleotides and nucleosides.

41. A method as in claim 23, wherein the single analyte is a therapeutic agent.

42. A method as in claim 23, wherein the single analyte is a neurotransmitter.

43. A method as in claim 23, wherein the sample consists essentially of aggregates of size of from about 500 nm to about 20 microns.

44. A method as in claim 23, wherein the at least one aggregate comprises a plurality of metal particles each having a dimension of no more than about 100 nm.

45. A method as in claim 23, wherein the at least one aggregate comprises a plurality of metal particles each having a dimension of no more than about 75 nm.

46. A method for determining the presence of an analyte, comprising:
providing a sample comprising a plurality of aggregates adsorbing a plurality of analytes, wherein each aggregate comprises a plurality of metal particles, each metal particle having a dimension of no more than about 100 nm and at least one aggregate adsorbs only one analyte;
exposing the sample to electromagnetic radiation to cause surface-enhanced emission;
obtaining spectral information of the sample, wherein the only one analyte contributes to the spectral information; and
determining the presence of the only one analyte from the spectral information.

47. A method as in claim 46, wherein the exposing step involves causing surface-enhanced emission and the obtaining step involves obtaining Raman spectral information.

48. A method as in claim 46, wherein the sample is free of an emission-enhancing aid.

49. A method as in claim 46, wherein the spectral information is a surface-enhanced Raman spectrum, having an enhancement factor of at least 1010.

50. A method as in claim 46, wherein the metal particles are selected from the group consisting of silver, gold and copper particles.

51. A method as in claim 46, wherein the aggregate is formed in situ by exposure to the electromagnetic radiation.

52. A method as in claim 46, wherein the plurality of aggregates is selected from the group consisting of a colloids suspended in a medium, aggregates deposited on a substrate and lithography produced metal aggregates.

53. A method as in claim 52, wherein the medium is selected from the group consisting of water, an organic solvent and a gel.

54. A method as in claim 52, wherein the substrate is selected from the group consisting of an electrode, a glass layer and a quartz layer.

55. A method as in claim 46, each metal particle having a dimension of no more than about 75 nm.

56. A method as in claim 46, wherein the electromagnetic radiation is non-resonant radiation.

57. A method as in claim 56, wherein the electromagnetic radiation is near infrared radiation.

58. A method as in claim 46, wherein the spectral information consists essentially of less than 5 lines of a Raman spectrum.

59. A method as in claim 46, wherein the single analyte is a dye.

60. A method as in claim 46, wherein the single analyte is selected from the group consisting of thymine, adenine, cytosine, guanine, and uracil.

61. A method as in claim 46, wherein the single analyte is selected from the group consisting of nucleotides and nucleosides.

62. A method as in claim 46, wherein the single analyte is a therapeutic agent.

63. A method as in claim 46, wherein the single analyte is a neurotransmitter.

64. A method for determining the presence of at least one analyte, comprising:
providing a sample comprising a plurality of aggregates, at least one aggregate adsorbing only one analyte that is free of an emission-enhancing aid;
exposing the sample to electromagnetic radiation; and
obtaining a spectrum, wherein the only one analyte contributes to at least one signal of the spectrum.

65. A method as in claim 64, wherein the spectrum is a surface-enhanced Raman spectrum, having an enhancement factor of at least 1010.

66. A method as in claim 64, wherein each aggregate of the plurality of aggregates comprises a plurality of metal particles.

67. A method as in claim 66, wherein the metal particles are selected from the group consisting of silver, gold and copper particles.

68. A method as in claim 64, wherein the plurality of aggregates is formed in situ by exposure to the electromagnetic radiation.

69. A method as in claim 64, wherein the plurality of aggregates is selected from the group consisting of a colloids suspended in a medium, aggregates deposited on a substrate and lithography produced metal aggregates.

70. A method as in claim 69, wherein the medium is selected from the group consisting of water, an organic solvent and a gel.

71. A method as in claim 69, wherein the substrate is selected from the group consisting of an electrode, a glass layer and a quartz layer.

72. A method as in claim 64, wherein the at least one aggregate has a dimension of at least about 500 nm.

73. A method as in claim 64, wherein the single analyte is a dye.

74. A method as in claim 64, wherein the single analyte is selected from the group consisting of thymine, adenine, cytosine, guanine, and uracil.

75. A method as in claim 64, wherein the single analyte is selected from the group consisting of nucleotides and nucleosides.

76. A method as in claim 64, wherein the single analyte is a therapeutic agent.

77. A method as in claim 64, wherein the single analyte is a neurotransmitter.

78. A method for determining the presence of a single analyte, comprising:
providing a sample comprising a plurality of surfaces, a portion of the plurality of surfaces adsorbing only one analyte; and
exposing the sample to electromagnetic radiation to cause the sample to emit radiation such that the sample is free of photobleaching.

79. A method as in claim 78, wherein the plurality of surfaces comprises a plurality of aggregates.

80. A method as in claim 79, wherein the plurality of aggregates comprises a plurality of metal particles.

81. A method as in claim 80, wherein the metal particles are selected from the group consisting of silver, gold and copper particles.

82. A method as in claim 79, wherein the plurality of aggregates is selected from the group consisting of a colloids suspended in a medium, aggregates deposited on a substrate and lithography produced metal aggregates.

83. A method as in claim 82, wherein the medium is selected from the group consisting of water, an organic solvent and a gel.

84. A method as in claim 82, wherein the substrate is selected from the group consisting of an electrode, a glass layer and a quartz layer.

85. A method as in claim 78, wherein the plurality of surfaces comprises a plurality of aggregates of metal particles, each of the metal particles having a dimension of no more than about 100 nm.

86. A method as in claim 78, wherein the only one analyte is a dye.

87. A method as in claim 78, wherein the only one analyte is selected from the group consisting of thymine, adenine, cytosine, guanine, and uracil.

88. A method as in claim 78, wherein the only one analyte is selected from the group consisting of nucleotides and nucleosides.

89. A method as in claim 78, wherein the only one analyte is a therapeutic agent.

90. A method as in claim 78, wherein the only one analyte is a neurotransmitter.

91. A method for determining the presence of at least one molecule, comprising providing at least one molecule, exposing the at least one molecule to electromagnetic radiation to cause Raman scattering, obtaining Raman spectral information and determining the presence of the at least one molecule from at least one anti-Stokes line.

92. A method as in claim 91, wherein the at least one molecule is adsorbed on a plurality of surfaces.

93. A method as in claim 91, wherein the at least one analyte is exposed to non-resonant radiation.

94. A method as in claim 92, wherein the electromagnetic radiation is near infrared radiation.

95. A method as in claim 94, wherein the near infrared radiation has a wavelength of at least 1000 nm.

96. A method for sequencing at least a portion of DNA or RNA, comprising:
cleaving the at least a portion of DNA or RNA into DNA or RNA fragments, wherein each fragment comprises at least one base;
allowing each DNA or RNA fragment to become surface-adsorbed;
exposing each fragment to electromagnetic radiation to cause surface-enhanced emission; and
obtaining unique surface-enhanced spectral information attributed to each fragment.

97. A method as in claim 96, wherein each fragment is surface-adsorbed onto one of a plurality of surfaces.

98. A method as in claim 97, wherein the plurality of surfaces is included in a moving stream.

99. A method as in claim 97, wherein the plurality of surfaces is selected from the group consisting of a plurality of aggregates suspended in a medium, a plurality of aggregates deposited on a substrate and lithography produced metal aggregates.

100. A method as in claim 99, wherein the plurality of aggregates comprise clusters of metal particles.

101. A method as in claim 100, wherein the metal particles are selected from the group consisting of silver, gold and copper particles.

102. A method as in claim 99, wherein the medium is selected from the group consisting of water, an organic solvent and a gel.

103. A method as in claim 100, wherein the substrate is selected from the group consisting of an electrode, a glass layer and a quartz layer.

104. A method as in claim 96, comprising allowing each fragment to become surface-absorbed on a plurality of protrusions and voids on a rough metal film.

105. A method as in claim 96, wherein the electromagnetic radiation is non-resonant radiation.

106. A method as in claim 96, wherein the electromagnetic radiation is near infrared radiation.

107. A method for general field enhancement, comprising providing a plurality of aggregates, exposing the plurality of aggregates to near infrared radiation and inducing at least one electromagnetic resonance in the plurality of aggregates to cause a surface-enhanced radiation.

108. A method as in claim 107, wherein the near infrared radiation has a wavelength of at least 1000 nm.

109. A method as in claim 107, wherein the plurality of aggregates comprises a plurality of metal particles.

110. A method as in claim 109, wherein the metal particles are selected from the group consisting of silver, gold and copper particles.

111. A method as in claim 107, wherein the aggregate is formed in situ by exposure to the electromagnetic radiation.

112. A method as in claim 107, wherein the plurality of aggregates is selected from the group consisting of colloids suspended in a medium, aggregates deposited on a substrate and lithography produced metal aggregates.

113. A method as in claim 112, wherein the medium is selected from the group consisting of water, an organic solvent and a gel.

114. A method as in claim 112, wherein the substrate is selected from the group consisting of an electrode, a glass layer and a quartz layer.

115. A method as in claim 109, wherein each metal particle has a dimension of no more than about 100 nm.

116. A method as in claim 109, wherein the plurality of aggregates comprises at least seven metal particles.

117. A method as in claim 107, wherein the surface enhanced radiation has an enhancement factor of at least 1010.

118. A method for selecting a spectral range, comprising:
providing a sample;
positioning at least one filter in association with an optical excitation and detection system, wherein the system is free of a spectrograph and the optical excitation system produces electromagnetic radiation in a first range;
exposing the sample to electromagnetic radiation via the system; and
obtaining a Raman spectrum of the sample having a second range wherein the second range is shifted from the first range.

119. A method as in claim 118, involving positioning at least two filters in association with the optical excitation and detection system.

120. A method as in claim 118, the positioning step involving positioning the at least one filter between a sample and detector of a Raman spectral system.

121. A method as in claim 118, wherein the second range is narrower than the first range.

122. A method for determining the presence of an analyte, comprising:
providing a sample comprising a rough metal film including a plurality of protrusions and indentations;
absorbing a plurality of analytes on a surface of the film;
exposing the sample to electromagnetic radiation to cause Raman scattering; and
obtaining a unique Raman signal attributed to a single analyte.

123. A system for determining the presence of at least one analyte, comprising:
a sample;
a source of electromagnetic radiation positioned to irradiate the sample; and
a detector positioned to detect surface-enhanced emission from the sample, wherein the sample comprises a plurality of aggregates of size of at least about 500 nm.

124. A system as in claim 123, wherein the sample comprises a plurality of aggregates of size of at least about 500 nm on a substrate.

125. A method comprising:
a) sequentially removing nucleotides from one end of at least one nucleic acid;
b) attaching each nucleotide to at least one nanoparticle;
c) identifying said nucleotides; and
d) determining the sequence of said nucleic acid.

126. The method of claim 125, wherein said nucleic acid is attached to a surface.

127. The method of claim 125, wherein said nanoparticles comprise a modified surface.

128. The method of claim 125, wherein said nucleotides are identified by surface enhanced Raman spectroscopy (SERS) and/or surface enhanced resonance Raman spectroscopy (SERRS).

129. The method of claim 125, wherein said nanoparticles comprise gold and/or silver.

130. The method of claim 129, wherein each nucleotide is attached to a single nanoparticle or a nanoparticle aggregate.

131. The method of claim 125, further comprising separating said nucleotides from said nucleic acid molecule.

132. The method of claim 128, further comprising exciting said nucleotides with a laser.

133. The method of claim 132, wherein a charge coupled device (CCD) camera is used to identify said nucleotides.

134. The method of claim 125, further comprising recording the identity of each nucleotide and the time at which each nucleotide is identified.

135. The method of claim 125, wherein an exonuclease is used to remove said nucleotides from said nucleic acid.

136. The method of claim 125, wherein said nanoparticles are between 10 nm and 20 micrometers in diameter.

137. The method of claim 136, wherein said nanoparticles are about 100 nm in diameter.

138. A method comprising:
a) obtaining nucleotides that are attached to Raman labels;
b) providing a nucleic acid comprising labeled nucleotides;
c) removing nucleotides from one end of the nucleic acid;
d) identifying nucleotides by Raman spectroscopy; and
e) determining the sequence of the nucleic acid.

139. The method of claim 138, further comprising passing the nucleotides removed from the nucleic acid in a stream.

140. The method of claim 138, wherein each type of nucleotide is labeled with a Raman label.

141. The method of claim 138, comprising labeling thymine.

142. The method of claim 138, comprising labeling adenine.

143. The method of claim 138, comprising labeling cytosine.

144. The method of claim 138, comprising labeling guanine.

145. The method of claim 138, comprising labeling uracil.

146. The method of claim 138, wherein said nucleotides are removed from said nucleic acid by exonuclease activity.

147. The method of claim 146, wherein only one nucleic acid at a time is exposed to exonuclease activity.

148. The method of claim 138, wherein said nucleotides are identified by surface enhanced Raman spectroscopy (SERS) and/or surface enhanced resonance Raman spectroscopy (SERRS).

149. The method of claim 148, further comprising attaching said nucleotides to nanoparticles.

150. An apparatus comprising:
a) a reaction site for immobilizing a DNA fragment onto an aggregate;
b) a first channel carrying a liquid stream, the first channel in fluid communication with said reaction site;
c) a second channel carrying the liquid stream, the second channel in fluid communication with said first channel;
d) a detection site in fluid communication with said first and second channels; and
e) a detection unit operably coupled to said detection site.

151. The apparatus of claim 150, wherein said detection unit comprises a Raman detector.

152. The apparatus of claim 151, wherein said detection unit comprises a laser and a CCD camera.

153. A method comprising:
a) sequentially removing nucleotides from one end of at least one nucleic acid;
b) moving the nucleotides in a stream packed with nanoparticles;
c) identifying the nucleotides by Raman spectroscopy; and
d) determining the sequence of the nucleic acid.

154. The method of claim 153, wherein the nucleotides are removed from the nucleic acid by exonuclease activity.

155. The method of claim 153, further comprising attaching said nucleic acid to a surface.

156. The method of claim 155, wherein said nucleic acid is immobilized in a reaction site.

157. The method of claim 156, wherein a single nucleic acid is immobilized in said reaction site.

158. The method of claim 153, wherein said nucleotides are identified by surface enhanced Raman spectroscopy (SERS) and/or surface enhanced resonance Raman spectroscopy (SERRS).

159. The method of claim 153, wherein at least two nanoparticles are cross-linked together.

160. The method of claim 153, wherein the nanoparticles comprise gold and/or silver, said nanoparticles between about 10 nm and 20 micrometers in size.

161. The method of claim 160, wherein the size of said nanoparticles is selected from the group consisting of about 10 to 50 nm, about 10 to 100 nm, about 10 nm and about 500 nm.

162. A method comprising:
a) preparing a nucleic acid comprising labeled nucleotides;
b) sequentially removing nucleotides from one end of the nucleic acid;
c) moving the nucleotides in a stream packed with nanoparticles;
d) identifying the nucleotides by Raman spectroscopy; and
e) determining the sequence of the nucleic acid.

163. The method of claim 162, wherein said nucleotides are labeled with one or more Raman labels.

164. The method of claim 163, wherein each type of nucleotide is labeled with a distinguishable Raman label.

165. The method of claim 163, comprising labeling thymine.

166. The method of claim 163, comprising labeling adenine.

167. The method of claim 163, comprising labeling cytosine.

168. The method of claim 163, comprising labeling guanine.

169. The method of claim 163, comprising labeling uracil.

170. The method of claim 162, further comprising separating said nucleotides from said nucleic acid.

171. The method of claim 162, further comprising recording the time at which each nucleotide passes through said channel.

172. The method of claim 13, wherein each type of nucleotide produces a unique Raman signal.

173. An apparatus comprising:
a) a reaction site for immobilizing a DNA fragment onto an aggregate;
b) a first channel carrying a liquid stream, the first channel in fluid communication with said reaction site;
c) a second channel carrying the liquid stream, the second channel in fluid communication with said first channel;
d) a multiplicity of nanoparticles in said second channel; and
e) a Raman detector operably coupled to said second channel.

174. The apparatus of claim 173, wherein said Raman detector comprises a laser and/or a CCD camera.

175. The apparatus of claim 173, further comprising a first electrode and a second electrode, said electrodes to move nucleotides from said first channel into said second channel.

176. The apparatus of claim 173, wherein said nanoparticles are cross-linked together.

177. The apparatus of claim 176, wherein said cross-linked nanoparticles provide an enhanced Raman signal.

178. The apparatus of claim 173, said detector to detect nucleotides by surface enhanced Raman spectroscopy (SERS) and/or surface enhanced resonance Raman spectroscopy (SERRS).

179. A method comprising:
a) removing one or more nucleotides from a nucleic acid;
b) attaching each of the one or more nucleotides to at least one nanoparticle;
c) identifying said nucleotides; and
d) determining the sequence of said nucleic acid.

180. The method of claim 179, wherein said nanoparticles comprise a modified surface.

181. The method of claim 179, wherein said nucleotides are identified by surface enhanced Raman spectroscopy (SERS) and/or surface enhanced resonance Raman spectroscopy (SERRS).

182. The method of claim 179, wherein said nanoparticles comprise gold and/or silver.

183. The method of claim 179, wherein each nucleotide is attached to a single nanoparticle or a nanoparticle aggregate.

184. The method of claim 179, further comprising exciting said nucleotides with a laser.

185. The method of claim 184, wherein a charge coupled device (CCD) camera is used to identify said nucleotides.

186. The method of claim 179, further comprising recording the identity of each nucleotide and the time at which each nucleotide is identified.

187. The method of claim 179, wherein said nanoparticles are between 10 nm and 20 micrometers in diameter.

188. A method comprising:
a) removing one or more nucleotides from a nucleic acid;
b) identifying each of the one or more nucleotides by Raman spectroscopy; and
c) determining the sequence of the nucleic acid.

189. The method of claim 188, wherein each type of nucleotide is labeled with a Raman label.

190. The method of claim 188, comprising labeling thymine.

191. The method of claim 188, comprising labeling adenine.

192. The method of claim 188, comprising labeling cytosine.

193. The method of claim 188, comprising labeling guanine.

194. The method of claim 188, comprising labeling uracil.

195. The method of claim 188, wherein said nucleotides are identified by surface enhanced Raman spectroscopy (SERS) and/or surface enhanced resonance Raman spectroscopy (SERRS).
